(19) United States
(12) Patent Application Publication
(10) Pub. No.: US 2021/0252251 A1
(43) Pub. Date: Aug. 19, 2021

(54) METHODS AND SYSTEMS FOR TREATMENT OF INSOMNIA USING DEEP BRAIN STIMULATION

(71) Applicant: Boston Scientific Neuromodulation Corporation, Valencia, CA (US)

(72) Inventor: Hari Hara Subramanian, Valencia, CA (US)

(21) Appl. No.: 17/177,649

(22) Filed: Feb. 17, 2021

Related U.S. Application Data
(60) Provisional application No. 62/978,546, filed on Feb. 19, 2020.

(51) Int. Cl. A61M 21/02; A61N 1/36; A61N 1/05

(52) U.S. Cl. CPC A61M 21/02; A61N 1/36082; A61M 2021/0072; A61N 1/36135; A61N 1/36146; A61N 1/0534

(57) ABSTRACT

A method of treating insomnia includes providing an electrical stimulation lead implanted into a brain of a patient, wherein the electrical stimulation lead includes a plurality of electrodes and at least one of the electrodes is disposed adjacent to or within a global pallidus externa of the patient; and delivering electrical stimulation to the global pallidus externa through at least one of the electrodes to treat insomnia.

### CROSS-REFERENCE TO RELATED APPLICATIONS

This application claims the benefit under 35 U.S.C. § 119(e) of U.S. Provisional Patent Application Ser. No. 62/978,546, filed Feb. 19, 2020, which is incorporated herein by reference.

### FIELD

The present disclosure is directed to the area of methods and systems for treatment of insomnia by electrical stimulation. The present disclosure is also directed to methods and systems for treatment of insomnia using deep brain stimulation of the global pallidus externa (GPe).

### BACKGROUND

Implantable electrical stimulation systems have proven therapeutic in a variety of diseases and disorders. For example, deep brain stimulation systems have been used as a therapeutic modality for the treatment of Parkinson's disease, essential tremor, and the like.

Stimulators have been developed to provide therapy for a variety of treatments. A stimulator can include an implantable pulse generator (IPG), one or more leads, and an array of stimulator electrodes on each lead. The stimulator electrodes are in contact with or near the nerves, muscles, or other tissue to be stimulated. The pulse generator in the IPG generates electrical pulses that are delivered by the electrodes to body tissue.

### BRIEF SUMMARY

One aspect is a method of treating insomnia that includes providing an electrical stimulation lead implanted into a brain of a patient, wherein the electrical stimulation lead includes a plurality of electrodes and at least one of the electrodes is disposed adjacent to or within a global pallidus externa of the patient; and delivering electrical stimulation to the global pallidus externa through at least one of the electrodes to treat insomnia.

In at least some aspects, the method further includes monitoring a blood biomarker and modifying stimulation parameters in response to the monitored blood biomarker. In at least some aspects, the blood biomarker is at least one of cortisol, melatonin, or serotonin. In at least some aspects, the method further includes halting stimulation after the blood biomarker achieves a threshold level.

In at least some aspects, the method further includes monitoring an electroencephalogram (EEG) of the patient and modifying stimulation parameters in response to the EEG. In at least some aspects, the method further includes monitoring movement of the patient and halting stimulation upon indication of locomotion of the patient.

In at least some aspects, the method further includes halting stimulation after a predetermined amount of time. In at least some aspects, the method further includes receiving user input to either start or stop electrical stimulation. In at least some aspects, the method further includes receiving user input to alter a set of stimulation parameters used to deliver the electrical stimulation. In at least some aspects, the method further includes receiving user input of a start time for delivery of the electrical stimulation. In at least some aspects, the method further includes receiving user input of a length of time for delivery of the electrical stimulation. In at least some aspects, the method further includes receiving user input of an end time for delivery of the electrical stimulation.

Another aspect is a method of programming an electrical stimulation system to treat insomnia. The method includes providing an electrical stimulation lead implanted into a brain of a patient, wherein the electrical stimulation lead includes a plurality of electrodes and at least one of the electrodes is disposed adjacent to or within a global pallidus externa of the patient; delivering electrical stimulation to the global pallidus externa through at least one of the electrodes using a first set of stimulation parameters to treat insomnia; observing a bioindicator to determine an effect of the electrical stimulation; and, when the bioindicator fails to reach a threshold, modifying the first set of stimulation parameters to form a modified set of stimulation parameters and repeating b) to d) using the modified set of stimulation parameters instead of the first set of stimulation parameters.

In at least some aspects, observing a bioindicator includes monitoring a blood biomarker. In at least some aspects, the blood biomarker is at least one of cortisol, melatonin, or serotonin. In at least some aspects, the threshold is a threshold expression of the blood biomarker. In at least some aspects, the method further includes halting stimulation after the blood biomarker achieves a second threshold level.

In at least some aspects, observing a bioindicator includes monitoring an electroencephalogram (EEG) of the patient. In at least some aspects, providing the electrical stimulation lead includes implanting the electrical stimulation lead into the brain of the patient.

In at least some aspects, the method further includes receiving user input of a start time for delivery of the electrical stimulation. In at least some aspects, the method further includes receiving user input of a length of time for delivery of the electrical stimulation. In at least some aspects, the method further includes receiving user input of an end time for delivery of the electrical stimulation.

A further aspect is a system for treating insomnia. The method includes an electrical stimulation lead including a plurality of electrodes and configured for implantation in a brain of a patient with at least one of the electrodes disposed adjacent to or within a global pallidus externa of the patient; and a control module couplable to the electrical stimulation lead and configured for: receiving programming instructions for the generation of electrical stimulation signals configured to recruit neurons in the global pallidus externa to treat insomnia; and generating the electrical stimulation signals.

In at least some aspects, the system further includes a sensor configured for monitoring a blood biomarker. In at least some aspects, the blood biomarker is at least one of cortisol, melatonin, or serotonin. In at least some aspects, the control module is also configured for halting stimulation after the blood biomarker achieves a threshold level. In at least some aspects, the control module is also configured for modifying stimulation parameters in response to the monitored blood biomarker. In at least some aspects, the control module is also configured for modifying the first set of stimulation parameters to form a modified set of stimulation parameters when the bioindicator fails to reach a threshold.

In at least some aspects, the control module is also configured for modifying one or more parameters of the electrical stimulation in response to an EEG. In at least some aspects, the system further includes a sensor configured for monitoring movement of the patient and halting stimulation upon indication of locomotion of the patient.

In at least some aspects, the control module is also configured for halting stimulation after a predetermined amount of time. In at least some aspects, the control module is also configured for receiving user input to either start or stop electrical stimulation. In at least some aspects, the control module is also configured for receiving user input of a start time for delivery of the electrical stimulation. In at least some aspects, the control module is also configured for receiving user input of a length of time for delivery of the electrical stimulation. In at least some aspects, the control module is also configured for receiving user input of an end time for delivery of the electrical stimulation.

Yet another aspect is a method for programming an implantable pulse generator of an electrical stimulation system. The method includes providing the implantable pulse generator of the electrical stimulation system; and programming the implantable pulse generator to generate electrical stimulation signals configured to recruit neurons in the global pallidus externa.

In at least some aspects, the method further includes coupling an electrical stimulation lead including a plurality of electrodes to the control module.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes one or more leads that can be coupled to an IPG;

FIG. 2 is a schematic view of another embodiment of an electrical stimulation system that includes a percutaneous lead coupled to an IPG;

FIG. 3 is a schematic view of one embodiment of a plurality of connector assemblies disposed in the IPG of FIG. 2, the connector assemblies configured and arranged to receive the proximal portions of the leads of FIG. 2;

FIG. 4 is a schematic view of one embodiment of a proximal portion of the lead of FIG. 2, a lead extension, and the IPG of FIG. 2, the lead extension configured and arranged to couple the lead to the IPG;

FIG. 5A is a schematic perspective view of a portion of one embodiment of a lead with thirty-two electrodes;

FIG. 5B is a schematic perspective view of portions of one embodiment of a lead with sixteen electrodes;

FIG. 5C is a schematic perspective view of portions of another embodiment of a lead with sixteen electrodes;

FIG. 5D is a schematic perspective view of portions of a third embodiment of a lead with sixteen electrodes;

FIG. 5E is a schematic perspective view of a portion of another embodiment of a lead with thirty-two electrodes;

FIG. 6 is a schematic overview of one embodiment of components of an electrical stimulation system;

FIG. 7 is a flowchart of one embodiment of method of treating insomnia; and

FIG. 8 is a flowchart of one embodiment of a method of programming an electrical stimulation system to treat insomnia.

### DETAILED DESCRIPTION

The present disclosure is directed to the area of methods and systems for treatment of insomnia by electrical stimulation. The present disclosure is also directed to methods and systems for treatment of insomnia using deep brain stimulation of the global pallidus externa (GPe).

Suitable implantable electrical stimulation systems include, but are not limited to, a least one lead with one or more electrodes disposed along a distal end of the lead and one or more terminals disposed along the one or more proximal ends of the lead. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties.

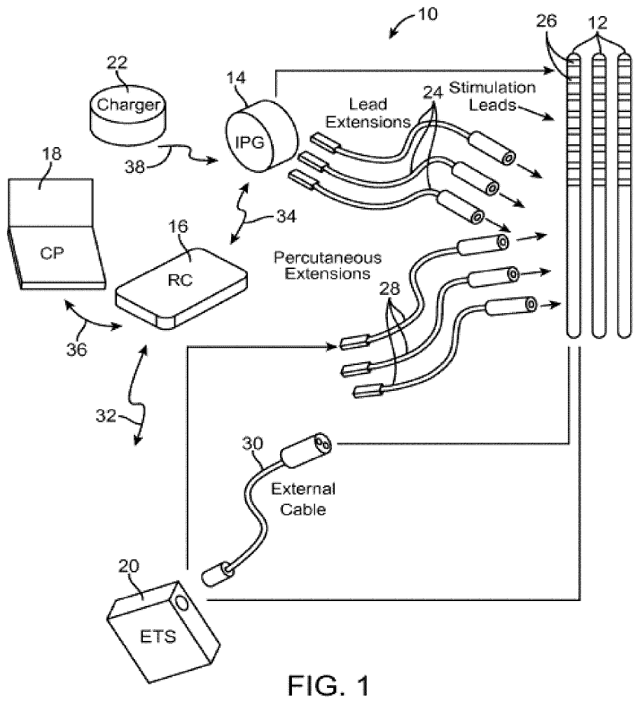

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG and ETS are examples of control modules for the electrical stimulation system.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's abdominal cavity or at any other suitable site. The implantable pulse generator 14 can have multiple stimulation channels which may be independently programmable to control the magnitude of the current stimulus from each channel. In some embodiments, the implantable pulse generator 14 can have any suitable number of stimulation channels including, but not limited to, 4, 6, 8, 12, 16, 32, or more stimulation channels. The implantable pulse generator 14 can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programmed via wireless communications (e.g., Bluetooth) between the RC 16 (or external device such as a hand-held electronic device like a mobile phone, tablet, or the like) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

Additional examples of the RC 16, CP 18, ETS 20, and external charger 22 can be found in the references cited herein as well as U.S. Pat. Nos. 6,895,280; 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, all of which are incorporated herein by reference in their entireties.

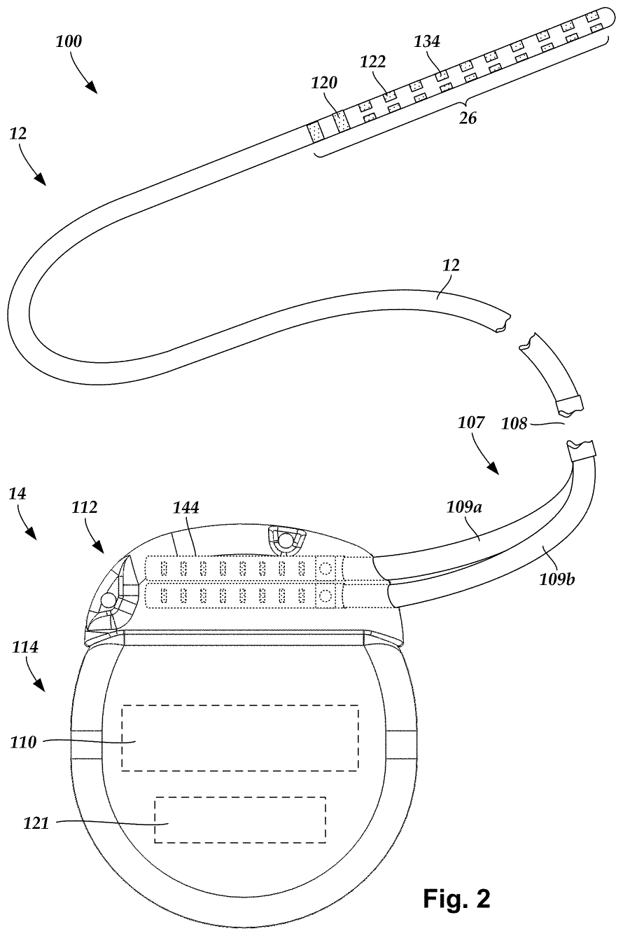

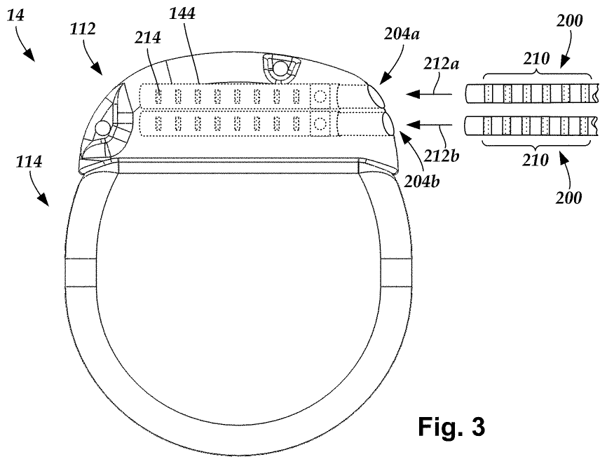

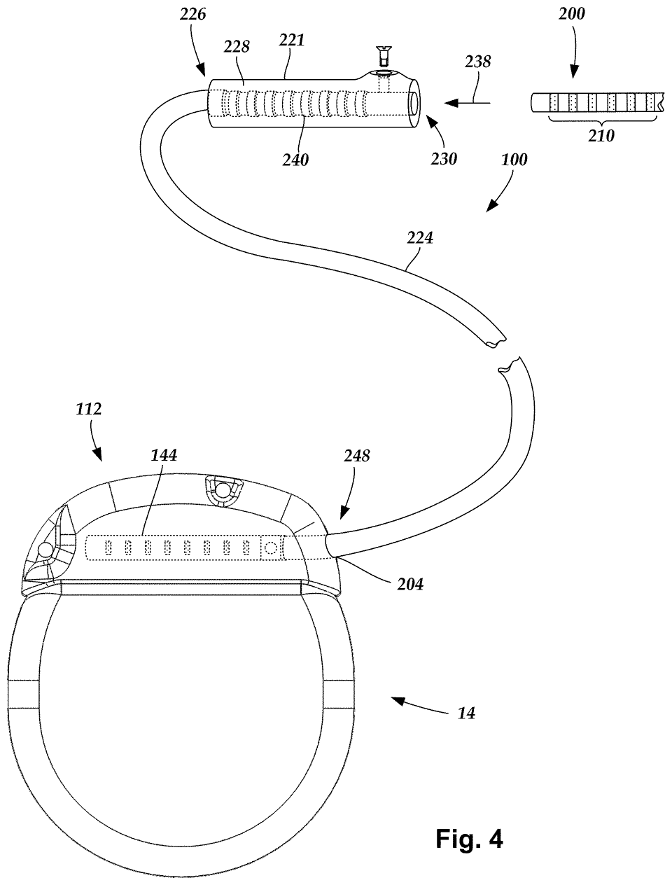

FIG. 2 illustrates schematically another embodiment of an electrical stimulation system 10. The electrical stimulation system includes an IPG (e.g., a control module) 14 and at least one lead 12 couplable to the IPG 14. The lead 12 includes one or more lead bodies 106, an array 26 of electrodes, such as electrode 134, and an array of terminals (e.g., 210 in FIGS. 3 and 4) disposed along the one or more lead bodies 106. In at least some embodiments, the lead is isodiametric along a longitudinal length of the lead body 106. FIG. 2 illustrates one lead 12 coupled to an IPG 14. Other embodiments may include two, three, four, or more leads 12 coupled to the IPG 14.

The lead 12 can be coupled to the IPG 14 in any suitable manner. In at least some embodiments, the lead 12 couples directly to the IPG 14. In at least some other embodiments, the lead 12 couples to the IPG 14 via one or more intermediate devices. For example, in at least some embodiments one or more lead extensions 224 (see e.g., FIG. 4) can be disposed between the lead 12 and the IPG 14 to extend the distance between the lead 12 and the IPG 14. Other intermediate devices may be used in addition to, or in lieu of, one or more lead extensions including, for example, a splitter, an adaptor, or the like or any combination thereof. It will be understood that, in the case where the electrical stimulation system 10 includes multiple elongated devices disposed between the lead 12 and the IPG 14, the intermediate devices may be configured into any suitable arrangement.

In FIG. 2, the electrical stimulation system 10 is shown having a splitter 107 configured and arranged for facilitating coupling of the lead 12 to the IPG 14. The splitter 107 includes a splitter connector 108 configured to couple to a proximal end of the lead 12, and one or more splitter tails 109a and 109b configured and arranged to couple to the IPG 14 (or another splitter, a lead extension, an adaptor, or the like).

In at least some embodiments, the IPG 14 includes a connector housing 112 and a sealed electronics housing 114. An electronic subassembly 110 and an optional power source 121 are disposed in the electronics housing 114. An IPG connector 144 is disposed in the connector housing 112. The IPG connector 144 is configured and arranged to make an electrical connection between the lead 12 and the electronic subassembly 110 of the IPG 14.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. Any number of electrodes 134 can be used for each array 26. For example, there can be two, four, six, eight, ten, twelve, fourteen, sixteen, or more electrodes 134. As will be recognized, other numbers of electrodes 134 may also be used.

The electrodes of the one or more lead bodies 106 are typically disposed in, or separated by, a non-conductive, biocompatible material such as, for example, silicone, polyurethane, polyetheretherketone (“PEEK”), epoxy, and the like or combinations thereof. The lead bodies 106 may be formed in the desired shape by any process including, for example, molding (including injection molding), casting, and the like. The non-conductive material typically extends from the distal end of the one or more lead bodies 106 to the proximal end of each of the one or more lead bodies 106.

Terminals (e.g., 210 in FIGS. 3 and 4) are typically disposed along the proximal end of the one or more lead bodies 106 of the electrical stimulation system 10 (as well as any splitters, lead extensions, adaptors, or the like) for electrical connection to corresponding connector contacts (e.g., 214 in FIGS. 3 and 240 in FIG. 4). The connector contacts are disposed in connectors (e.g., 144 in FIGS. 2 to 4; and 221 in FIG. 4) which, in turn, are disposed on, for example, the IPG 14 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the one or more lead bodies 106. In at least some embodiments, the one or more lumens are permanently or removably sealable at the distal end.

FIG. 3 is a schematic side view of one embodiment of a proximal end of one or more elongated devices 200 configured and arranged for coupling to one embodiment of the IPG connector 144. The one or more elongated devices may include, for example, the lead body 106, one or more intermediate devices (e.g., the splitter 107 of FIG. 2, the lead extension 224 of FIG. 4, an adaptor, or the like or combinations thereof), or a combination thereof. FIG. 3 illustrates two elongated devices 200 coupled to the IPG 14. These two elongated devices 200 can be two tails as illustrated in FIG. 2 or two different leads or any other combination of elongated devices.

The IPG connector 144 defines at least one port into which a proximal end of the elongated device 200 can be inserted, as shown by directional arrows 212a and 212b. In FIG. 3 (and in other figures), the connector housing 112 is shown having two ports 204a and 204b. The connector housing 112 can define any suitable number of ports including, for example, one, two, three, four, five, six, seven, eight, or more ports.

The IPG connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the IPG 14 to the electrodes (134 of FIG. 2) disposed at a distal end of the lead 12. Examples of connectors in IPGs are found in, for example, U.S. Pat. No. 7,244,150 and 8,224,450, which are incorporated by reference in their entireties.

FIG. 4 is a schematic side view of another embodiment of the electrical stimulation system 10. The electrical stimulation system 10 includes a lead extension 224 that is configured and arranged to couple one or more elongated devices 200 (e.g., the lead body 106, the splitter 107, an adaptor, another lead extension, or the like or combinations thereof) to the IPG 14. In FIG. 4, the lead extension 224 is shown coupled to a single port 204 defined in the IPG connector 144. Additionally, the lead extension 224 is shown configured and arranged to couple to a single elongated device 200. In alternate embodiments, the lead extension 224 is configured and arranged to couple to multiple ports 204 defined in the IPG connector 144, or to receive multiple elongated devices 200, or both.

A lead extension connector 221 is disposed on the lead extension 224. In FIG. 4, the lead extension connector 221 is shown disposed at a distal end 226 of the lead extension 224. The lead extension connector 221 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 2) disposed along the lead (12 in FIG. 2).

In at least some embodiments, the proximal end of the lead extension 224 is similarly configured and arranged as a proximal end of the lead 12 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 4), the proximal end 248 of the lead extension 224 is configured and arranged for insertion into the IPG connector 144.

Returning to FIG. 2, in at least some embodiments at least some of the stimulation electrodes take the form of segmented electrodes that extend only partially around the perimeter (for example, the circumference) of the lead. These segmented electrodes can be provided in sets of electrodes, with each set having electrodes circumferentially distributed about the lead at a particular longitudinal position.

In FIG. 2, the electrodes 134 are shown as including both ring electrodes 120 and segmented electrodes 122. In some embodiments, the electrodes 134 are all segmented electrode or all ring electrodes. The segmented electrodes 122 of FIG. 2 are in sets of three (one of which is not visible in FIG. 2), where the three segmented electrodes of a particular set are electrically isolated from one another and are circumferentially offset along the lead 12. Any suitable number of segmented electrodes can be formed into a set including, for example, two, three, four, or more segmented electrodes. The lead 12 of FIG. 2 has thirty segmented electrodes 122 (ten sets of three electrodes each) and two ring electrodes 120 for a total of 32 electrodes 134.

Segmented electrodes can be used to direct stimulus current to one side, or even a portion of one side, of the lead. When segmented electrodes are used in conjunction with an implantable pulse generator that delivers current stimulus, current steering can be achieved to more precisely deliver the stimulus to a position around an axis of the lead (i.e., radial positioning around the axis of the lead). Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a segmented electrode array, current steering can be performed not only along a length of the lead but also around a perimeter of the lead. This provides precise three-dimensional targeting and delivery of the current stimulus to neural target tissue, while potentially avoiding stimulation of other tissue.

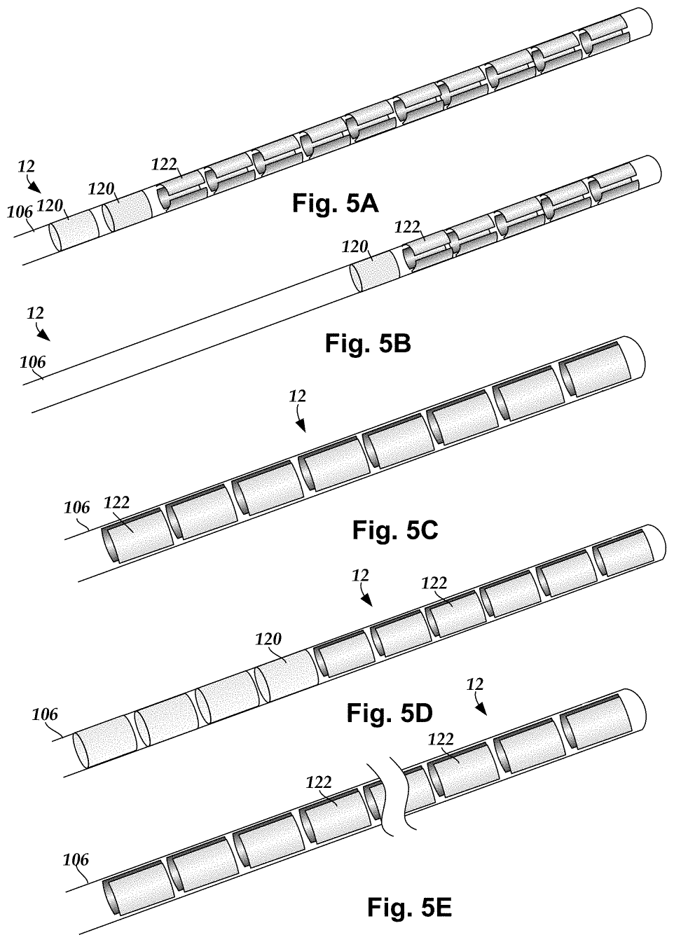

FIG. 5A illustrates a 32-electrode lead 12 with a lead body 106 and two ring electrodes 120 proximal to thirty segmented electrodes 122 arranged in ten sets of three segmented electrodes each. In the illustrated embodiments, the ring electrodes 120 are proximal to the segmented electrodes 122. In other embodiments, one or more of the ring electrodes 120 can be proximal to, or distal to, one or more of the segmented electrodes 122.

Any number of segmented electrodes 122 may be disposed on the lead body including, for example, one, two, three, four, five, six, seven, eight, nine, ten, eleven, twelve, thirteen, fourteen, fifteen, sixteen, twenty, twenty-four, twenty-eight, thirty, thirty-two, or more segmented electrodes 122. It will be understood that any number of segmented electrodes 122 may be disposed along the length of the lead body. A segmented electrode 122 typically extends only 75%, 67%, 60%, 50%, 40%, 33%, 25%, 20%, 17%, 15%, or less around the circumference of the lead.

The segmented electrodes 122 may be grouped into sets of segmented electrodes, where each set is disposed around a circumference of the lead 12 at a particular longitudinal portion of the lead 12. The lead 12 may have any number of segmented electrodes 122 in a given set of segmented electrodes. The lead 12 may have one, two, three, four, five, six, seven, eight, or more segmented electrodes 122 in a given set. The lead 12 may have any number of sets of segmented electrodes including, but not limited to, one, two, three, four, five, six, eight, ten, twelve, fifteen, sixteen, twenty, or more sets. The segmented electrodes 122 may be uniform, or vary, in size and shape. In some embodiments, the segmented electrodes 122 are all of the same size, shape, diameter, width or area or any combination thereof. In some embodiments, the segmented electrodes 122 of each circumferential set (or even all segmented electrodes disposed on the lead 12) may be identical in size and shape.

Each set of segmented electrodes 122 may be disposed around the circumference of the lead body to form a substantially cylindrical shape around the lead body. The spacing between individual electrodes of a given set of the segmented electrodes may be the same, or different from, the spacing between individual electrodes of another set of segmented electrodes on the lead 12. In at least some embodiments, equal spaces, gaps or cutouts are disposed between each segmented electrode 122 around the circumference of the lead body. In other embodiments, the spaces, gaps or cutouts between the segmented electrodes 122 may differ in size or shape. In other embodiments, the spaces, gaps, or cutouts between segmented electrodes 122 may be uniform for a particular set of the segmented electrodes 122, or for all sets of the segmented electrodes 122. The sets of segmented electrodes 122 may be positioned in irregular or regular intervals along a length of the lead body.

FIG. 5B to 5E illustrate other embodiments of leads with segmented electrodes 122. FIG. 5B illustrates a sixteen electrode lead 12 having one ring electrode 120 that is proximal to five sets of three segmented electrodes 122 each. FIG. 5C illustrates a sixteen electrode lead 12 having eight sets of two segmented electrodes 122 each. As illustrated in FIG. 5C, an embodiment of a lead 12 does not necessarily include a ring electrode. FIG. 5D illustrates a sixteen electrode lead 12 having four ring electrodes 120 that are proximal to six sets of two segmented electrodes 122 each. FIG. 5E illustrates a thirty-two electrode lead 12 having sixteen sets of two segmented electrodes 122 each (for clarity of illustration, not all of the electrodes are shown). It will be recognized that any other electrode combination of ring electrodes, segmented electrodes, or both types of electrodes can be used.

When the lead 12 includes both ring electrodes 120 and segmented electrodes 122, the ring electrodes 120 and the segmented electrodes 122 may be arranged in any suitable configuration. For example, when the lead 12 includes two or more ring electrodes 120 and one or more sets of segmented electrodes 122, the ring electrodes 120 can flank the one or more sets of segmented electrodes 122. Alternately, the two or more ring electrodes 120 can be disposed proximal to the one or more sets of segmented electrodes 122 or the two or more ring electrodes 120 can be disposed distal to the one or more sets of segmented electrodes 122 or any other suitable arrangement of the ring electrodes 120 and segmented electrodes 122.

The electrodes 120, 122 may have any suitable longitudinal length including, but not limited to, 2, 3, 4, 4.5, 5, or 6 mm. The longitudinal spacing between adjacent electrodes 120, 122 may be any suitable amount including, but not limited to, 1, 2, or 3 mm, where the spacing is defined as the distance between the nearest edges of two adjacent electrodes. In some embodiments, the spacing is uniform between longitudinally adjacent of electrodes along the length of the lead. In other embodiments, the spacing between longitudinally adjacent electrodes may be different or non-uniform along the length of the lead.

Examples of leads with segmented electrodes include U.S. Patent Application Publications Nos. 2010/0268298; 2011/0005069; 2011/0078900; 2011/0130803; 2011/0130816; 2011/0130817; 2011/0130818; 2011/0078900; 2011/0238129; 2011/0313500; 2012/0016378; 2012/0046710; 2012/0071949; 2012/0165911; 2012/0197375; 2012/0203316; 2012/0203320; 2012/0203321; 2013/0197602; 2013/0261684; 2013/0325091; 2013/0317587; 2014/0039587; 2014/0353001; 2014/0358209; 2014/0358210; 2015/0018915; 2015/0021817; 2015/0045864; 2015/0021817; 2015/0066120; 2013/0197424; 2015/0151113; 2014/0358207; and U.S. Pat. No. 8,483,237, all of which are incorporated herein by reference in their entireties. A lead may also include a tip electrode and examples of leads with tip electrodes include at least some of the previously cited references, as well as U.S. Patent Application Publications Nos. 2014/0296953 and 2014/0343647, all of which are incorporated herein by reference in their entireties. A lead with segmented electrodes may be a directional lead that can provide stimulation in a particular direction using the segmented electrodes.

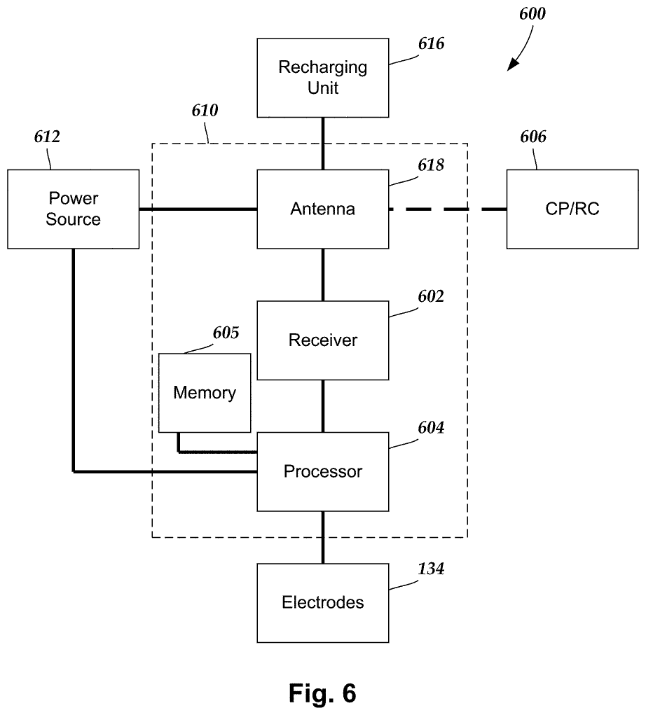

FIG. 6 is a schematic overview of one embodiment of components of an electrical stimulation system 600 including an electronic subassembly 610 disposed within an IPG. It will be understood that the electrical stimulation system can include more, fewer, or different components and can have a variety of different configurations including those configurations disclosed in the stimulator references cited herein.

Some of the components (for example, power source 612, antenna 618, receiver 602, processor 604, and memory 605) of the electrical stimulation system can be positioned on one or more circuit boards or similar carriers within a sealed housing of an implantable pulse generator, if desired. Any power source 612 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the optional antenna 618 or a secondary antenna. The external power source can be in a device that is mounted on the skin of the user or in a unit that is provided near the user on a permanent or periodic basis.

If the power source 612 is a rechargeable battery, the battery may be recharged using the optional antenna 618, if desired. Power can be provided to the battery for recharging by inductively coupling the battery through the antenna to a recharging unit 616 external to the user. Examples of such arrangements can be found in the references identified above.

In one embodiment, electrical current is emitted by the electrodes 134 on the lead body to stimulate nerve fibers, muscle fibers, or other body tissues near the electrical stimulation system. A processor 604 is generally included to control the timing and electrical characteristics of the electrical stimulation system. For example, the processor 604 can, if desired, control one or more of the timing, frequency, amplitude, width, and waveform of the pulses. In addition, the processor 604 can select which electrodes can be used to provide stimulation, if desired. In some embodiments, the processor 604 may select which electrode(s) are cathodes and which electrode(s) are anodes. In some embodiments, the processor 604 may be used to identify which electrodes provide the most useful stimulation of the desired tissue. Instructions for the processor 604 can be stored on the memory 605.

Any processor can be used and can be as simple as an electronic device that, for example, produces pulses at a regular interval or the processor can be capable of receiving and interpreting instructions from the CP/RC 606 (such as CP 18 or RC 16 of FIG. 1) that, for example, allows modification of pulse characteristics. In the illustrated embodiment, the processor 604 is coupled to a receiver 602 which, in turn, is coupled to the optional antenna 618. This allows the processor 604 to receive instructions from an external source to, for example, direct the pulse characteristics and the selection of electrodes, if desired.

In one embodiment, the antenna 618 is capable of receiving signals (e.g., RF signals) from a CP/RC 606 (see, CP 18 or RC 16 of FIG. 1) which is programmed or otherwise operated by a user. The signals sent to the processor 604 via the antenna 618 and receiver 602 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 600 to cease operation, to start operation, to start charging the battery, or to stop charging the battery. In other embodiments, the stimulation system does not include an antenna 618 or receiver 602 and the processor 604 operates as programmed.

Optionally, the electrical stimulation system 600 may include a transmitter (not shown) coupled to the processor 604 and the antenna 618 for transmitting signals back to the CP/RC 606 or another unit capable of receiving the signals. For example, the electrical stimulation system 600 may transmit signals indicating whether the electrical stimulation system 600 is operating properly or not or indicating when the battery needs to be charged or the level of charge remaining in the battery. The processor 604 may also be capable of transmitting information about the pulse characteristics so that a user or clinician can determine or verify the characteristics.

Insomnia disorder (ID) is a debilitating sleep disorder with negative effects on health and quality of life, including the risk of suicide. ID may also be a symptom of other diseases or disorders, such as Parkinson's disease. Medications to treat insomnia often produce substantial side-effects, such as deleterious effects on the autonomic system, including, for example, liver dysfunction, renal-angiotensin mediated cardiac ischemia, depression, and possibly tumerogenesis.

The global pallidus externa (GPe) is a component of the basal ganglia that is thought to play a role in regulating sleep and mental behavior. Although no particular theory or hypothesis is necessary to practice the present invention, it is thought that dopamine, acting on D2 receptors on the striatopallidal terminals, enhances activity in the GPe and promotes sleep. Thus, the GPe can be an effective target site for the treatment or amelioration of insomnia via deep brain stimulation (DBS) therapy.

To treat insomnia, an electrical stimulation lead can be implanted in the GPe or adjacent to the GPe to stimulate a portion of the GPe. In at least some embodiments, a system can provide electrical stimulation to recruit neurons in the global pallidus externa to treat insomnia. Because the GPe is a relatively large brain structure (for example, 10-12 mm in length), in at least some embodiments, a lead with 32 electrodes, such as the leads illustrated in FIGS. 5A and 5E may be used. The large number of electrodes of such leads allow for coverage of a larger region of tissue, as well as focusing to very specific tissue regions by selection of one or more of the electrodes for providing stimulation. It will be understood, however, that any of the other leads disclosed herein, as well as other leads and electrode arrangements, can be used for stimulation of the GPe to treat insomnia or to recruit neurons in the global pallidus externa to treat insomnia.

When the lead is implanted, electrical stimulation can be performed through the electrodes of the lead using one or more sets of electrical stimulation parameters which define the delivery of the stimulation. The electrical stimulation parameters may control various parameters of the stimulation current applied to a stimulation site including, but not limited to, the frequency, pulse width, amplitude, waveform (e.g., square or sinusoidal), electrode configuration (i.e., anode-cathode assignment), burst pattern (e.g., continuous or intermittent), duty cycle or burst repeat interval, ramp on time, and ramp off time. Specific stimulation parameters may have different effects and side-effects. Thus, in at least some embodiments, the stimulation parameters may be adjusted to facilitate or enhance treatment.

In at least some embodiments, unilateral stimulation of the GPe using a lead implanted in or near the GPe is performed to treat insomnia. In other embodiments, bilateral stimulation can be performed in which the GPe is stimulated using a first lead to treat insomnia and a second lead (or other electrodes on the first lead) are used to stimulate another brain structure to also treat insomnia or to treat another disease, disorder, or symptoms (for example, a movement disorder, depression, or the like.) In at least some embodiments, the two leads may be implanted in different hemispheres of the brain. In at least some embodiments, the two leads can be coupled to the same IPG using, for example, different ports of the IPG.

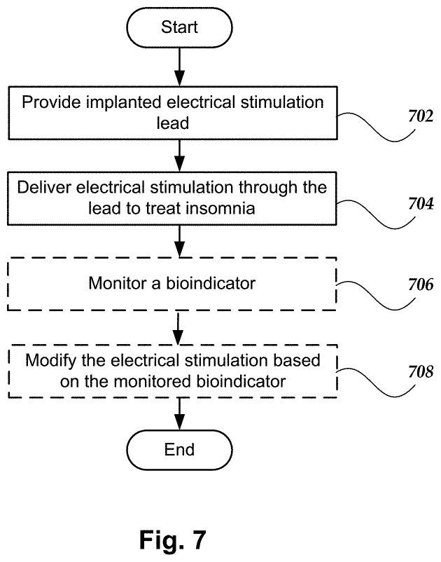

FIG. 7 is a flowchart of one embodiment of a method of treating insomnia. In step 702, an implanted electrical stimulation lead is provided in or near the GPe. For example, the electrical stimulation lead may be implanted into the brain of the patient so that at least some of the electrodes are disposed in or near the GPe. Following implantation, a programming process may be used to determine a set of stimulation parameters for the treatment as discussed below. The IPG may also be implanted. In at least some embodiments, the IPG is implanted in the torso with the lead, or a lead extension coupled to the lead, extending under the skin to the IPG. In at least some embodiments, the lead can be coupled instead to an ETS or other external stimulator.

In step 704, electrical stimulation is delivered through the lead to treat insomnia using the set of stimulation parameters. In at least some embodiments, a system recruits neurons in the global pallidus externa to treat insomnia. In at least some embodiments, the patient or a clinician may be able to alter the stimulation parameter using, for example, a CP or RC. In at least some embodiments, the electrical stimulation is delivered during a sleep period or prior to (for example, 5, 10, 15, 30, 45, 60, or 90 minutes prior to) a sleep period or both. In at least some embodiments, the sleep period (for example, the duration or start time or stop time or any combination thereof) may be programmed into the IPG, CP, or RC. In at least some embodiments, a user, such as the patient, may trigger the start or end of the sleep period or electrical stimulation. In at least some embodiments, the user may use a RC or CP to turn on or off the electrical stimulation. In at least some embodiments, the IPG, RC, CP, or other device in communication with the IPG, RC, or CP includes a sensor, such as a gyroscope or accelerometer, that monitors movement (e.g., locomotor movement) of the patient and may be configured to halt stimulation when the IPG, RC, or CP detects a threshold amount of movement indicating that the patient is awake.

Optionally, in step 706, a bioindicator can be monitored. The monitoring can be periodic, aperiodic, random, or the like. The bioindicator can be any suitable indicator that is associated with sleep or insomnia. For example, the level of one or more blood biomarkers, such as cortisol, melatonin, or serotonin, can be indicative of sleep, sleepiness, or insomnia. Accordingly, monitoring one or more of the blood biomarkers can be indicative of one or more of the following: 1) level of sleepiness, 2) level of insomnia, 3) entry into, or depth of, sleep, or any combination thereof. In at least some embodiments, effective electrical stimulation may also be indicated by a change (for example, an increase) in one or more of the blood biomarkers. This change may be expected to occur over time such as, for example, over a period of 5, 10, 15, 20, 30, 45, 60, or 90 minutes or more. Other bioindicators can include an electroencephalogram (EEG), such as, for example, a cortical EEG, or the like. The bioindicator may be monitored using any suitable arrangement or technique including, but not limited to, a biochemical or EEG sensor which is optionally associated with or in communication with the IPG, RC, or CP; a biochemical testing protocol utilizing blood or other body fluid (e.g., sweat) sampling protocol; or the like or any combination thereof.

Optionally, in step 708, the electrical stimulation may be modified based on the monitored bioindicator. For example, the electrical stimulation may be modified by halting stimulation (for example, if the stimulation is effective and the bioindicator suggests that the patient is sleeping) or by altering one or more of the electrical stimulation parameters (for example, to increase or decrease the effectiveness of the stimulation as indicated by the monitored bioindicator.) Any suitable electrical stimulation parameter can be altered including, but not limited to, amplitude, pulse width, electrode selection, duty cycle, or the like or any combination thereof. In at least some embodiments, the monitoring of the bioindicator can be part of a feedback loop to dynamically or adaptively adjust the stimulation parameters or delivery of stimulation on a periodic, aperiodic, random, or continuous basis according to the patient state indicated by the monitored bioindicator.

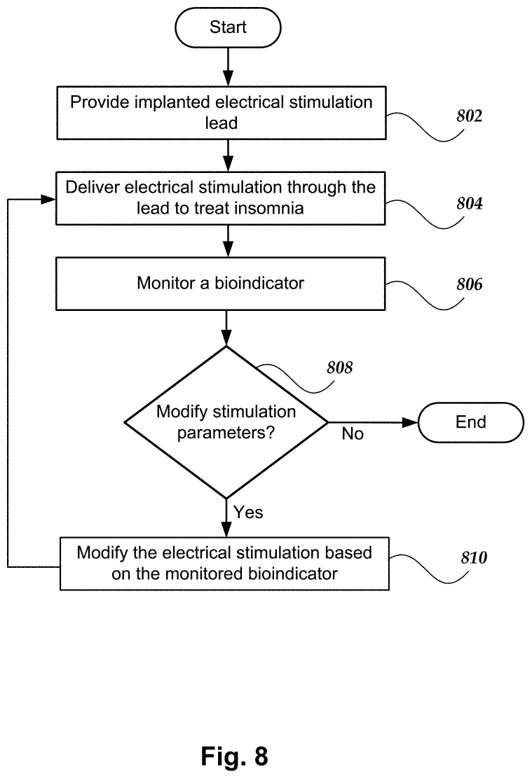

In addition to providing electrical stimulation, the bioindicator(s) can be used to facilitate programming of the electrical stimulation system (for example, the IPG.) FIG. 8 is a flowchart of one embodiment of a method of programming an electrical stimulation system to treat insomnia. In step 802, an implanted electrical stimulation lead is provided in or near the GPe. For example, the electrical stimulation lead may be implanted into the brain of the patient so that at least some of the electrodes are disposed in or near the GPe. The IPG may also be implanted. Often the IPG is implanted in the torso with the lead, or a lead extension coupled to the lead, extending under the skin to the IPG. In some embodiments, the lead can be coupled to an ETS or other external stimulator.

In step 804, a set of stimulation parameters is used to deliver electrical stimulation through the lead to treat insomnia using the set of stimulation parameters. In at least some embodiments, a system recruits neurons in the global pallidus externa to treat insomnia. The initial set of stimulation parameters can be selected by a user, such as a clinician, or may be a default set of stimulation parameters or may be selected in any other manner. The programmer may also program a sleep period.

In step 806, as the patient is stimulated a bioindicator is monitored. The monitoring can be periodic, aperiodic, random, or the like. The bioindicator can be any suitable indicator that is associated with sleep or insomnia, such as one or more blood biomarkers, an electroencephalogram (EEG), such as, for example, a cortical EEG, or the like, as described above. The bioindicator may be monitored using any suitable arrangement or technique including, but not limited to, a biochemical or EEG sensor which is optionally associated with or in communication with the IPG, RC, or CP; a biochemical testing protocol utilizing blood or other body fluid (e.g., sweat) sampling protocol; or the like or any combination thereof.

In step 808, it is determined by the programmer or electrical stimulation system whether to adjust or modify the stimulation parameters. If no, then the programming ends and, if yes, then the programming proceeds to step 810. For example, the programmer or electrical stimulation system may determine whether the stimulation is adequate or sufficient (for example, the electrical stimulation provides a threshold level of treatment of insomnia.) In at least some embodiments, if the electrical stimulation fails to produce a threshold level of a bioindicator then the programmer or system can proceed to step 810 to adjust or modify the stimulation parameters.

In step 810, one or more of the stimulation parameters of the set of electrical stimulation parameters can be altered or modified based on the monitored bioindicator. Any suitable electrical stimulation parameter can be altered or modified including, but not limited to, amplitude, pulse width, electrode selection, duty cycle, or the like or any combination thereof. In at least some embodiments, the programmer (for example, the patient or a clinician may be able to alter the stimulation parameter using, for example, a CP or RC. In at least some embodiments, the monitoring of the bioindicator can be part of a feedback loop to dynamically or adaptively adjust the stimulation parameters or delivery of stimulation on a periodic, aperiodic, or continuous basis according to the patient state indicated by the monitored bioindicator.

After modifying one or more of the stimulation parameters, the process returns to step 804 and can continue in a loop of steps 804 to 810 until the process ends or the programmer or system terminates the process.

It will be understood that each block of the flowchart illustration, and combinations of blocks in the flowchart illustration and methods disclosed herein, can be implemented by computer program instructions. In addition, the feature extraction engine, storage engine, visualization engine, and storage programming engine may be implemented by computer program instructions. These program instructions may be provided to a processor to produce a machine or engine, such that the instructions, which execute on the processor, create means for implementing the actions specified in the flowchart block or blocks or engine disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device. The computer program instructions can be stored locally or nonlocally (for example, in the Cloud).

The above specification and examples provide a description of the arrangement and use of the invention. Since many embodiments of the invention can be made without departing from the spirit and scope of the invention, the invention also resides in the claims hereinafter appended.

What is claimed as new and desired to be protected is:

1. A method of treating insomnia, the method comprising:
providing an electrical stimulation lead implanted into a brain of a patient, wherein the electrical stimulation lead comprises a plurality of electrodes and at least one of the electrodes is disposed adjacent to or within a global pallidus externa of the patient; and
delivering electrical stimulation to the global pallidus externa through at least one of the electrodes to treat insomnia.

2. The method of claim 1, further comprising monitoring a blood biomarker and modifying stimulation parameters in response to the monitored blood biomarker.

3. The method of claim 2, wherein the blood biomarker is at least one of cortisol, melatonin, or serotonin.

4. The method of claim 2, further comprising halting stimulation after the blood biomarker achieves a threshold level.

5. The method of claim 1, further comprising monitoring an electroencephalogram (EEG) of the patient and modifying stimulation parameters in response to the EEG.

6. The method of claim 1, further comprising monitoring movement of the patient and halting stimulation upon indication of locomotion of the patient.

7. The method of claim 1, further comprising halting stimulation after a predetermined amount of time.

8. The method of claim 1, further comprising receiving user input to either start or stop electrical stimulation.

9. The method of claim 1, further comprising receiving user input to alter a set of stimulation parameters used to deliver the electrical stimulation.

10. The method of claim 1, further comprising receiving user input of a start time for delivery of the electrical stimulation.

11. The method of claim 1, further comprising receiving user input of a length of time for delivery of the electrical stimulation.

12. The method of claim 1, further comprising receiving user input of an end time for delivery of the electrical stimulation.

13. A method of programming an electrical stimulation system to treat insomnia, the method comprising:
providing an electrical stimulation lead implanted into a brain of a patient, wherein the electrical stimulation lead comprises a plurality of electrodes and at least one of the electrodes is disposed adjacent to or within a global pallidus externa of the patient;
delivering electrical stimulation to the global pallidus externa through at least one of the electrodes using a first set of stimulation parameters to treat insomnia;
observing a bioindicator to determine an effect of the electrical stimulation; and
when the bioindicator fails to reach a threshold, modifying the first set of stimulation parameters to form a modified set of stimulation parameters and repeating b) to d) using the modified set of stimulation parameters instead of the first set of stimulation parameters.

14. The method of claim 13, wherein observing a bioindicator comprises monitoring a blood biomarker and modifying stimulation parameters in response to the monitored blood biomarker.

15. The method of claim 14, wherein the blood biomarker is at least one of cortisol, melatonin, or serotonin.

16. The method of claim 14, wherein the threshold is a threshold expression of the blood biomarker.

17. The method of claim 14, further comprising halting stimulation after the blood biomarker achieves a second threshold level.

18. The method of claim 13, wherein observing a bioindicator comprises monitoring an electroencephalogram (EEG) of the patient and modifying stimulation parameters in response to the EEG.

19. The method of claim 13, wherein providing the electrical stimulation lead comprises implanting the electrical stimulation lead into the brain of the patient.

20. The method of claim 1, further comprising receiving user input of a start time or an end time for delivery of the electrical stimulation.
